(19) United States
(12) Patent Application Publication
(10) Pub. No.: US 2010/0209364 A1
(43) Pub. Date: Aug. 19, 2010

(54) Emulsified Composition

(75) Inventors: Masamichi Abe, Osaka-shi (JP); Takashi Ishikawa, Osaka-shi (JP); Takashi Katano, Osaka-shi (JP); Junko Marukawa, Osaka-shi (JP); Shinya Kasuga, Osaka-shi (JP)

Correspondence Address: THE WEBB LAW FIRM, P.C., 700 KOPPERS BUILDING, 436 SEVENTH AVENUE, PITTSBURGH, PA, 15219, US

(73) Assignee: ROHTO PHARMACEUTICAL CO., LTD., Osaka-shi (JP)

(21) Appl. No.: 12/669,744

(22) PCT Filed: Jul. 17, 2008
(86) PCT No.: PCT/JP2008/062912

§ 371 Date: Jan. 19, 2010

(30) Foreign Application Priority Data
Jul. 20, 2007 (JP) 2007-190216

(51) Int. Cl. A61K 8/55; A61K 31/685; A61Q 17/04; A61P 17/00

(52) U.S. Cl. 424/59; 514/78

(57) ABSTRACT

An emulsified composition comprises (A) petrolatum, (B) lecithin, (C) glycerin, (D) water and (E) a water-soluble polymer, wherein the content of the petrolatum (A) is 10 to 30 wt % relative to 100 wt % of the emulsified composition, and an internal phase of the emulsified composition has an average particle diameter of not more than 800 nm as measured by a dynamic light scattering method based on the dynamic light scattering theory.

### FIELD OF THE INVENTION

The present invention relates to emulsified compositions that comprise petrolatum, glycerin, lecithin, water and water-soluble polymer and show excellent moisture-retaining properties, good usability and high storage stability.

### BACKGROUND OF THE INVENTION

In the treatment, prevention or improvement of symptoms caused by skin dryness, petrolatum has been used as a base to increase skin occlusion and to decrease evaporation of water from the skin. However, because the petrolatum cannot contain water, it cannot supply water to the skin. Further, the petrolatum is semisolid and is hence hard to apply to a large area of the skin showing dry symptoms. Furthermore, greasy feeling after applying it is significant and therefore has a problem in usability.

To solve such problems, emulsified compositions such as emulsions and creams are used as bases containing petrolatum. In the production of such emulsified compositions, surfactants are essentially used. However, many of the generally used surfactants show skin irritation. Furthermore, the blending amount of petrolatum should be increased in order to achieve sufficient skin occlusion and effect of decreasing evaporation of water from the skin sufficiently; however, this requires that the amounts of surfactants necessary for emulsification are increased.

Skin showing dry symptom has lowered its barrier function and is sensitive to external stimuli. Emulsified compositions (bases) applied to users having such a sensitive skin condition should have less skin irritation. To this end, it would be effective to use surfactants with low skin irritation or reduce the amount of surfactants used. However, surfactants showing low skin irritation often have low emulsifying power and the obtainable emulsion system is frequently unstable. Reducing the amount of surfactants has similarly encountered with a problem of lowered emulsion stability.

These problems as described above have been diligently addressed. For example, Patent Document 1 discloses an emulsified composition obtained using petrolatum in a large blending amount, glycerin, a less skin irritating surfactant such as lecithin, and a gelling agent such as polycarboxypolyvinyl. The invention of Patent Document 1 involves inert mineral powder such as kaolin, talc or calcium carbonate to increase the emulsion stability of the emulsified composition. However, the emulsion stability is insufficient even after addition of them. Further, the addition of the powder results in rough texture and the like, and improvements in sense of use are also required.

Patent Documents 2 and 3 also disclose emulsified compositions obtained using petrolatum in a large blending amount, glycerin and a less skin irritating surfactant such as lecithin. The emulsified composition of Patent Document 2 has a problem in storage stability under severe conditions as described in Patent Document 3. The emulsified composition of Patent Document 3 achieves increased storage stability by further containing betaine as a surfactant. However, as described in Patent Document 4, increasing the blending amount of betaine causes greasiness and is not desirable in view of sense of use.

In the treatment, prevention or improvement of symptoms caused by skin dryness, polyhydric alcohols such as glycerin are generally used as moisturizers due to their excellent moisture-retaining properties. However, Patent Document 5 discloses that the polyhydric alcohols attract ticks (e.g., Dermatophagoides farinae and Dermatophagoides pteronyssinus) which can evoke allergic dermatitis or atopic dermatitis. The patent document indicates that the ticks cause allergic dermatitis or atopic dermatitis and worsen the symptoms, and are also a factor that induces skin dryness. Accordingly, emulsified compositions that repel the ticks have been desired.

### Patent Document 1: JP-A-H02-31834

### Patent Document 2: JP-A-2001-72581

### Patent Document 3: JP-A-2003-95956

### Patent Document 4: JP-A-H08-133947

### Patent Document 5: JP-A-H09-176006

### SUMMARY OF THE INVENTION

It is an object of the invention to provide emulsified compositions which are capable of increasing skin occlusion, have good sense of use while containing a sufficient amount of petrolatum enough to decrease the evaporation of water from the skin, can supply sufficient water to the skin, have sustainable and high moisture-retaining properties, and show low irritation and high storage stability. It is another object of the invention to provide emulsified compositions that repel ticks.

The present inventors have invented compositions which comprise (B) lecithin as a less skin irritating surfactant, (A) 10 to 30 wt % of petrolatum, (C) glycerin, (D) water and (E) a water-soluble polymer and in the internal phase of which has an average particle diameter of not more than 800 nm as measured by a dynamic light scattering method based on the dynamic light scattering theory. The emulsified compositions have been found to have good sense of use while containing a sufficient amount of petrolatum enough to decrease the evaporation of water from the skin, be capable of supplying sufficient water to the skin, have sustainable and high moisture-retaining properties, and show low irritation and high storage stability. It has also been found that the emulsified compositions repel ticks although they contain glycerin having a tick-attracting effect. The present invention has been completed based on the findings.

The present invention provides the following emulsified compositions.

[1] An emulsified composition which comprises (A) 10 to 30 wt % of petrolatum, (B) lecithin, (C) glycerin, (D) water and (E) a water-soluble polymer and an internal phase of which has an average particle diameter of not more than 5000 nm.

[2] The emulsified composition as described in [1], which comprises (A) petrolatum, (B) lecithin, (C) glycerin, (D) water and (E) a water-soluble polymer, wherein the content of the petrolatum (A) is 10 to 30 wt % relative to 100 wt % of the emulsified composition, and the internal phase of the emulsified composition has an average particle diameter of not more than 800 nm as measured by a dynamic light scattering method based on the dynamic light scattering theory.

[3] The emulsified composition as described in [1] or [2], wherein the water-soluble polymer (E) is at least one polymer selected from the group consisting of cellulose polymers, vinyl polymers, acrylic acid polymers, plant polymers, microbial polymers, phospholipid polar group-containing polymers and mucopolysaccharides.

[4] The emulsified composition as described in any one of [1] to [3], wherein the content of the glycerin (C) is 10 to 20 wt % relative to 100 wt % of the emulsified composition.

[5] The emulsified composition as described in any one of [1] to [4], further comprising at least one active ingredient selected from the group consisting of nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents.

[6] The emulsified composition as described in any one of [1] to [4], wherein the composition further comprises at least one active ingredient selected from the group consisting of nonsteroidal anti-inflammatory drugs, steroids, local anesthetics, antipruritics and moisturizers.

[7] The emulsified composition as described in any one of [1] to [6], which is used to treat, prevent or improve a symptom caused by skin dryness and/or a disease that shows a symptom caused by skin dryness.

[8] A method for treating, preventing or improving a symptom caused by skin dryness and/or a disease that shows a symptom caused by skin dryness, comprising applying to skin an effective amount of the emulsified composition described in any one of [1] to [7].

[9] Use of the emulsified composition described in anyone of [1] to [7] in the manufacture of medicaments or cosmetics for treating, preventing or improving a symptom caused by skin dryness and/or a disease that shows a symptom caused by skin dryness.

### ADVANTAGEOUS EFFECTS OF THE INVENTION

Provided by the present invention is the novel emulsified compositions which have good sense of use while containing a sufficient amount of petrolatum enough to decrease the evaporation of water from the skin, can supply sufficient water to the skin, have sustainable and high moisture-retaining properties, show low irritation and high storage stability, and repel ticks although they contain glycerin having a tick-attracting effect.

The emulsified compositions of the present invention have excellent moisture-retaining properties and are therefore useful as compositions and the like for the treatment, prevention or improvement of a symptom caused by skin dryness and/or a disease that shows a symptom caused by skin dryness. Further, the emulsified compositions of the present invention possess a tick-repelling effect and are therefore useful as compositions for the treatment, prevention or improvement of symptoms caused by ticks such as allergic dermatitis and atopic dermatitis.

### BRIEF DESCRIPTION OF THE DRAWINGS

FIG. 1 is a set of micrographs of emulsified compositions of Example 2 and Comparative Example 5 applied to a glass plate (upper: Example 2, lower: Comparative Example 5).

FIG. 2 is a figure showing results of measurements of water content in horny layer in Test Example 5. In FIG. 2, the horizontal axis represents time (min) and the longitudinal axis represents conductance (μS). The five line graphs in FIG. 2 indicate the result of Example 1 by the rhombus mark, the result of Example 2 by the square mark, the result of Example 3 by the triangle mark, the result of Comparative Example 1 by the cross mark, and the result of Comparative Example 3 by the asterisk mark.

### PREFERRED EMBODIMENT OF THE INVENTION

The present invention will be described in detail hereinbelow. The terms used in the present specification shall be understood to have the meaning usually used in the field of art to which the present invention pertains, unless otherwise specified.

An emulsified composition according to the present invention comprises (A) petrolatum, (B) lecithin, (C) glycerin, (D) water and (E) a water-soluble polymer, in which the content of the petrolatum (A) is 10 to 30 wt % relative to 100 wt % of the emulsified composition, and the internal phase of the emulsified composition has an average particle diameter of not more than 800 nm as measured by a dynamic light scattering method based on the dynamic light scattering theory. The ingredients will be described below.

### [(A) Petrolatum]

The petrolatums (A) used in the present invention are semisolids obtained by purifying a mixture of hydrocarbons and any of those generally used in medicaments, quasi drugs and cosmetics can be used without limitation.

Yellow petrolatum or white petrolatum may be used in the present invention. From the viewpoint of low irritation and the like, white petrolatum with low impurity content is preferable.

The blending amount of the petrolatum in the present invention is 10 to 30 wt %, preferably 12 to 27 wt %, and particularly preferably 15 to 25 wt % based on the whole emulsified composition (100 wt %). If the blending amount of the petrolatum is less than 10 wt %, occlusion (reduction of water evaporation amount) is deteriorated as will be demonstrated in Test Examples later. If it exceeds 30 wt %, the sense of use is deteriorated and further, anticipated occlusion (reduction of water evaporation amount) cannot be achieved as will be demonstrated in Test Examples later.

From the viewpoints of emulsion stability and sense of use of the emulsified compositions of the present invention, the blending amount of the petrolatum (A) is preferably 13 to 75 wt %, and more preferably 16 to 45 wt % based on 100 wt % of water (D) in the present invention.

### [(B) Lecithin]

The lecithins (B) used in the present invention are mixtures of phospholipids such as phosphatidylcholine, phosphatidylethanolamine, phosphatidylserine and phosphatidylinositol, with components such as triglycerides, fatty acids and carbohydrates from vegetable oils. Their composition and physical properties greatly vary depending on origin, purification degree, chemical treatment and the like, but any lecithins generally used in medicaments, quasi drugs or cosmetics may be used without limitation.

The lecithins (B) used in the present invention may be natural lecithins of animal or plant origin (e.g., soy lecithin, egg yolk lecithin), lecithins that are obtained by chemical treatment of natural lecithins, or lecithins that have an increased phosphatidylcholine content by purifying natural lecithins with solvents such as acetone. The phosphatidylcholine content in the lecithins (B) used in the present invention is not particularly limited.

Examples of the lecithins obtained by chemical treatment of natural lecithins include hydrogenated lecithins obtained by hydrogenation treatment (e.g., completely hydrogenated lecithins, partially hydrogenated lecithins), and hydroxylated lecithins by obtained hydroxylation. The lecithins (B) used in the present invention may be lysolecithins from the natural lecithins or chemically treated lecithins. However, the lysolecithins are easily oxidized, so that the use of lysolecithins lowers the storage stability of the emulsified composition of the present invention and can result in irritation. Therefore, the lecithins are preferably not lysolecithins.

The lecithins (B) may be used singly, or two or more kinds may be used in combination.

Specific examples of the lecithins (B) used in the present invention include soy lecithin, egg yolk lecithin, purified soy lecithin, purified egg yolk lecithin, hydrogenated soy lecithin, egg yolk lysophosphatidylcholine and soy lysophospholipid.

The blending amount of the lecithins (B) in the present invention is not particularly limited as long as the advantageous effects of the present invention are achieved. In general, it is 0.1 to 5 wt %, preferably 0.5 to 3 wt %, and particularly preferably 1.2 to 2 wt % based on the whole emulsified composition. If the amount is less than 0.1 wt %, emulsification tends to be difficult. If it exceeds 5 wt %, smell or coloration can be caused.

Further, from the viewpoints of emulsion stability and sense of use of the emulsified composition of the present invention, the blending amount of the lecithins (B) in the present invention is preferably 5 to 30 wt %, and more preferably 10 to 20 wt % based on 100 wt % of the petrolatum (A).

Still further, from the viewpoints of emulsion stability and sense of use, the blending amount of the lecithins (B) in the present invention is preferably 0.1 to 13 wt %, and more preferably 0.7 to 5 wt % based on 100 wt % of water (D).

### [(C) Glycerin]

The glycerin (C) used in the present invention is not particularly limited, and any glycerin generally used in medicaments, quasi drugs or cosmetics may be used.

The blending amount of the glycerin (C) in the present invention is not particularly limited as long as the advantageous effects of the present invention are achieved. In general, it is 1 to 30 wt %, preferably 5 to 25 wt %, and particularly preferably 10 to 20 wt % based on the whole emulsified composition. If it is less than 1 wt %, moisture-retaining effects may be insufficient. If it exceeds 30 wt %, stickiness tends to increase.

Further, from the viewpoints of moisture-retaining effects of the emulsified composition of the present invention, the blending amount of the glycerin (C) in the present invention is preferably 20 to 200 wt %, and more preferably 30 to 150 wt % based on 100 wt % of the petrolatum (A).

Still further, from the viewpoint of moisture-retaining effects of the emulsified composition of the present invention, the blending amount of the glycerin (C) in the present invention is preferably 1.3 to 75 wt %, and more preferably 6.7 to 42 wt % based on 100 wt % of water (D).

### [(D) Water]

The emulsified composition of the present invention contains water (D). The blending amount of the water (D) in the present invention is not particularly limited as long as the advantageous effects of the present invention are achieved. In general, it is 40 to 75 wt %, preferably 60 to 75 wt %, and particularly preferably 60 to 70 wt % based on the whole emulsified composition.

Further, from the viewpoints of emulsion stability and sense of use of the emulsified composition of the present invention, the blending amount of the water (D) in the present invention is preferably 250 to 550 wt %, and more preferably 400 to 500 wt % based on 100 wt % of the petrolatum (A).

### [(E) Water-Soluble Polymer]

The water-soluble polymers (E) used in the present invention are not particularly limited, and any water-soluble polymers generally used in medicaments, quasi drugs or cosmetics may be used.

The water-soluble polymers (E) increase applicability and tick-repelling effect of the emulsified composition of the present invention.

The water-soluble polymers (E) may be in the form of salts. Exemplary salts of the water-soluble polymers include alkali metal salts such as sodium salts and potassium salts; and alkaline earth metal salts such as magnesium salts and calcium salts.

Specific examples of the water-soluble polymers (E) for use in the invention include:

cellulose polymers such as methyl cellulose, ethyl cellulose, carboxymethyl cellulose, hydroxyethyl cellulose, hydroxyethyl methyl cellulose, hydroxypropyl cellulose, hydroxypropyl methyl cellulose, carmellose sodium and stearoxy hydroxypropyl methyl cellulose;

vinyl polymers such as polyvinyl alcohol (partially saponified products thereof), polyvinylpyrrolidone, polyethylene glycol, carboxyvinyl polymer, polyvinyl methyl ether and ammonium N-acryloyldimethyltaurate/vinylpyrrolidone copolymer;

acrylic acid polymers such as sodium polyacrylate, partially neutralized polyacrylic acid and acrylic acid/alkyl methacrylate copolymers (such as Pemulen (registered trademark));

plant polymers such as gum arabic, tragacanth gum, galactan, guar gum, pectin, carrageenan, alginic acid, sodium alginate and propylene glycol alginate ester;

microbial polymers such as xanthan gum, dextran and pullulan;

mucopolysaccharides such as chondroitin sulfuric acid, sodium chondroitin sulfate, hyaluronic acid and sodium hyaluronate; and

phospholipid polar group-containing polymers such as MPC polymers (e.g., LIPIDURE (registered trademark)).

The water-soluble polymers (E) may be used singly, or two or more kinds may be used in combination.

Preferred water-soluble polymers (E) used in the present invention are:

polyvinyl alcohol (partially saponified products thereof), polyvinylpyrrolidone, polyethylene glycol, carboxyvinyl polymer, poly vinyl methyl ether, ammonium N-acryloyldimethyltaurate/vinylpyrrolidone copolymer;

sodium polyacrylate, partially neutralized polyacrylic acid, acrylic acid/alkyl methacrylate ester copolymers (such as Pemulen (registered trademark));

gum arabic, tragacanth gum, galactan, guar gum, pectin, carrageenan, alginic acid, sodium alginate;

xanthan gum, dextran, pullulan;

chondroitin sulfuric acid, sodium chondroitin sulfate, hyaluronic acid and sodium hyaluronate. In particular, carboxyvinyl polymer, acrylic acid/alkyl methacrylate ester copolymers (such as Pemulen (registered trademark)), propylene glycol alginate ester, xanthan gum, hyaluronic acid and sodium hyaluronate are preferred.

The blending amount of the water-soluble polymers (E) in the present invention is not particularly limited as long as the advantageous effects of the present invention are achieved. In general, it is 0.0001 to 5 wt %, preferably 0.001 to 3 wt %, more preferably 0.01 to 3 wt %, and particularly preferably 0.01 to 1 wt % based on the whole emulsified composition.

Further, from the viewpoints of emulsion stability and sense of use of the emulsified composition of the present invention, the blending amount of the water-soluble polymers (E) in the present invention is preferably 0.1 to 10 wt %, more preferably 1 to 7 wt %, and particularly preferably 2 to 4 wt % based on 100 wt % of the petrolatum (A).

Still further, from the viewpoints of emulsion stability and sense of use of the emulsified composition of the present invention, the blending amount of the water-soluble polymers (E) in the present invention is preferably 0.00013 to 12.5 wt %, and more preferably 0.0013 to 5 wt % based on 100 wt % of the water (D).

The emulsified composition of the present invention may contain additional active ingredients to achieve desired effects in the composition. The active ingredients in the present invention refer to components that have beneficial effects on skin, such as pharmacologically active components and physiologically active components. The active ingredients are not particularly limited. Examples of them include nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents. These components may be used singly, or two or more kinds may be used in combination.

Specifically, the following components can be exemplified.

nonsteroidal anti-inflammatory agents: glycyrrhizic acid derivatives such as Glycyrrhiza extract, glycyrrhizic acid, dipotassium glycyrrhizinate and monoammonium glycyrrhizinate; glycyrrhetinic acid or derivatives thereof; allantoin or derivatives thereof; indomethacin; ibuprofen; ibuprofen piconol; bufexamac; butyl flufenamate; bendazac; piroxicam; ketoprofen; felbinac; salicylic acid derivatives such as methyl salicylate and salicylic acid glycol; menthol; and camphor.

Vitamins: vitamins A such as retinol, retinol acetate, retinol palmitate, retinal, retinoic acid, methyl retinoate, ethyl retinoate, retinol retinoate, vitamin A fatty acid esters, d-δ-tocopheryl retinoate, α-tocopheryl retinoate and β-tocopheryl retinoate;

provitamins A such as β-carotene, α-carotene, γ-carotene, δ-carotene, lycopene, zeaxanthin, cryptoxanthin and echinenone;

vitamins E such as α-tocopherol, β-tocopherol, δ-tocopherol, tocopherol acetate, dl-α-tocopherol succinate and dl-α-tocopherol calcium succinate;

vitamins B2 such as riboflavin, flavin mononucleotide, flavin adenine dinucleotide, riboflavin butyrate, riboflavin tetrabutyrate, riboflavin 5′-phosphate sodium and riboflavin tetranicotinate;

nicotinic acids such as methyl nicotinate, nicotinic acid and nicotinamide;

vitamins C such as ascorbic stearate, L-ascorbyl dipalmitate, ascorbyl tetraisopalmitate, ascorbic acid, sodium ascorbate, dehydroascorbic acid, sodium ascorbic phosphate, magnesium ascorbic phosphate, magnesium ascorbic phosphate salt, sodium ascorbic phosphate salt and ascorbic acid glucoside;

vitamins D such as methyl hesperidin, ergocalciferol and cholecalciferol;

vitamins K such as phylloquinone and farnoquinone;

vitamins B1 such as γ-oryzanol, dibenzoyl thiamine, dibenzoyl thiamine hydrochloride, thiamine hydrochloride, thiamine cetyl hydrochloride, thiamine thiocyanate, thiamine lauryl hydrochloride, thiamine nitrate, thiamine monophosphate, lysine salt of thiamine, thiamine triphosphate, thiamine monophosphate phosphate salt, thiamine monophosphate, thiamine diphosphate, thiamine diphosphate hydrochloride, thiamine triphosphate and thiamine triphosphate monophosphate salt;

vitamins B6 such as pyridoxine hydrochloride, pyridoxine acetate, pyridoxal hydrochloride, pyridoxal 5′-phosphate and pyridoxamine hydrochloride;

vitamins B12 such as cyanocobalamin, hydroxocobalamin and deoxyadenosylcobalamin;

folic acids such as folic acid and pteroylglutamic acid;

pantothenic acids such as pantothenic acid, calcium pantothenate, pantothenyl alcohol (panthenol), D-pantethaine, D-pantethine, coenzyme A and pantothenyl ethyl ether;

biotins such as biotin and bioticyn;

vitamin-like acting factors such as carnitine, ferulic acid, α-lipoic acid and orotic acid;

whitening agents such as placenta; arbutin; cysteine; ellagic acid; kojic acid; phytic acid; rucinol; hydroquinone; and components, extracts and essential oils derived from plants such as iris, almond, aloe, ginkgo, oolong tea, rose fruit, Scutellaria root, Coptis, Hypericum erectum Thunberg, dead nettle, seaweed, Pueraria root, camomile, Glycyrrhiza, Gardenia, Sophora root, wheat, rice, rice germ, oryzanol, rice bran, Perilla frutescens, peony, Cnidium rhizome, Mulberry bark, soybean, tea, Terminalia, Angelica sinensis, Calendula officinalis Linne, hamamelis, safflower, moutan bark, Coix seeds, nettle tree, persimmon (Diospyros kaki) and clove;

anti-wrinkle agents such as ubiquinones such as coenzymes Q6 to Q10, kinetin, glycolic acid, argiline, acylated glucosamine, collagen, aloe extract, seaweed extract, horse chestnut extract, rosemary extract and extract of Rodgersia podophylla A. Gray;

anti-inflammatory analgesics such as indomethacin, felbinac, methyl salicylate, glycol salicylate, allantoin or its derivatives, ibuprofen, ibuprofen piconol, bufexamac, butyl flufenamate, bendazac, piroxicam and ketoprofen;

antifungal agents such as terbinafine hydrochloride, sulconazole nitrate, clotrimazole, isoconazole nitrate, croconazole nitrate, miconazole nitrate, econazole nitrate, oxiconazole nitrate, bifonazole, tioconazole, ketoconazole, tolnaftate, tolciclate, liranaftate, ciclopirox olamine, exalamide, siccanin, undecylenic acid, zinc undecylenate, pyrroInitrin, butenafine hydrochloride, amorolfine hydrochloride and neticonazole hydrochloride;

steroids such as dexamethasone valerate acetate, dexamethasone, dexamethasone propionate, dexamethasone acetate, dexamethasone valerate, prednisolone valerate acetate, hydrocortisone butyrate, hydrocortisone acetate, hydrocortisone, hydrocortisone butyrate propionate, cortisone acetate, prednisolone acetate, prednisolone, betamethasone, betamethasone valerate, betamethasone dipropionate, clobetasone butyrate, clobetasol propionate, diflorasone acetate, diflucortolone valerate, beclomethasone propionate, flumethasone pivalate, triamcinolone acetonide, fluocinolone acetonide, fluocinonide, amcinonide, halcinonide and difluprednate;

hair restorers such as procyanidin, dipotassium glycyrrhizinate, carpronium chloride, cepharanthine, menthol, hinokitiol, L-hydroxyproline, acetyl hydroxyproline, fucoidan, capsicum tincture, cepharanthine, swertianin, Ginseng extract, flavonosteroids, minoxidil, FGF-10, Isodon japonicus Hara extract (essence), Swertia extract (essence), Laminaria angustata extract (essence), Gynostemma pentaphyllum extract (essence), Hypericum erectum Thunberg extract (essence), gentian extract (essence), sage extract (essence), peppermint extract (essence), hop extract (essence), Coix seed extract (essence), persimmon leaf extract (essence), Rehmannia root extract (essence), carrot extract (essence), Tilia miqueliana extract (essence) and moutan bark extract (essence);

slimming agents such as xanthines such as caffeine, aminophylline, theophylline, oxtriphylline, dyphylline, diisobutylaminobenzoyloxypropyl theophylline, theobromine, diprophylline, proxyphylline and pentoxifylline; and capsaicin;

local anesthetics such as lidocaine, lidocaine hydrochloride, dibucaine, dibucaine hydrochloride, ethyl aminobenzoate, Eucalyptus oil, eugenol, camphor, peppermint oil and turpentine oil;

antipruritics such as crotamiton, chlorpheniramine, chlorpheniramine maleate, diphenhydramine, diphenhydramine hydrochloride, diphenhydramine salicylate, salicylic acid, nonylic acid vanillylamide, mequitazine, camphor, thymol, eugenol, polyoxyethylene lauryl ether, comfrey extract and Perilla frutescens extract;

antimicrobials such as isopropylmethylphenol, chlorhexidine gluconate, chlorhexidine hydrochloride, benzalkonium chloride, benzethonium chloride, cetyltrimethylammonium bromide, dequalinium chloride, triclosan and trichlorocarbanilide;

antivirals such as acyclovir and penciclovir;

keratin softeners such as ethyl alcohol, isopropyl alcohol, propanol, butanol, polyethylene glycol, benzyl alcohol, phenylethyl alcohol, propylene carbonate, hexyldodecanol, dimethylsulfoxide, dimethylacetamide, dimethylformamide, triethanolamine, diisopropyladipate, ethyl laurylate, lanolin, fatty acid dialkylolamide, urea, sulfur, resorcin, phytic acid, lactic acid, lactates, sodium hydroxide and potassium hydroxide;

moisturizers such as high-molecular compounds such as polyethylene glycol, diglycerin trehalose, heparinoid, collagen, elastin, keratin, chitin and chitosan; natural moisturizing factors such as amino acids such as glycine, aspartic acid and arginine, sodium lactate, urea and sodium pyrrolidone carboxylate; plant extracts such as camomile extract, Aloe extract, Aloe vera extract, hamamelis extract, rosemary extract, thyme extract, tea extract and Perilla frutescens extract; ceramides such as ceramide 1, ceramide 2, ceramide 3, ceramide 4, ceramide 5, ceramide 6I, ceramide 6II and ceramide 7; N-(hexadecyloxyhydroxypropyl)-N-hydroxyethyl decanamide and N-(hexadecyloxyhydroxypropyl)-N-hydroxyethyl hexadecanamide;

astringents such as citric acid, tartaric acid, lactic acid, aluminum chloride, aluminum sulfate, allantoin chlorohydroxyaluminum, allantoin dihydroxyaluminum, aluminum phenolsulfonate, zinc para-phenolsulfonate, zinc sulfate, zinc lactate and aluminum chlorohydroxide;

antioxidants such as dibutylhydroxytoluene, butylhydroxyanisole, disodium ethylenediaminetetraacetate dihydrate (hereinafter also referred to as sodium edetate), sorbic acid and sodium sulfite;

hair growth inhibitors such as isoflavone, blackberry lily extract, Houttuynia cordata extract, orris root extract and papain enzyme;

UV absorbents such as 2-ethylhexyl paramethoxycinnamate, hexyl 2-[4-(diethylamino)-2-hydroxybenzoyl]benzoate, 2,4,6-tris[4-(2-ethylhexyloxycarbonyl)anilino]-1,3,5-triazine, 2-ethylhexyl dimethoxybenzylidene oxoimidazolidinepropionate and 2,4-bis[[4-(2-ethylhexyloxy)-2-hydroxy]-phenyl]-6-(4-methoxyphenyl)-1,3,5-triazine; and

UV scattering agents such as inorganic compounds such as zinc oxide, titanium oxide, iron oxide, cerium oxide, zirconium oxide, titanium silicate, zinc silicate, silicic anhydride and cerium silicate; these inorganic compounds coated with inorganic powders such as mica and talc; particulate composites of these inorganic compounds with resins such as polyamides, polyethylenes, polyesters, polystyrenes and nylons; and these inorganic compounds treated with, for example, silicone oils or fatty acid aluminum salts.

Among the above ingredients, in view of the use of the emulsified compositions of the present invention for the treatment, prevention or improvement of skin diseases, it is preferable to blend at least one active ingredient selected from the group consisting of the nonsteroidal anti-inflammatory agents, steroids, local anesthetics, antipruritics and moisturizers into the emulsified composition of the present invention.

Among the above ingredients, in view of the application to atopic dermatitis for which it is important that the evaporation of water from the skin should be reduced, the emulsified composition of the present invention preferably contains steroids which are frequently used in the treatment of atopic dermatitis.

Of the steroids, prednisolone valerate acetate generally known as antedrug steroid is particularly preferred from the aspect of safety.

In view of the application to xeroderma for which it is likewise important that the evaporation of water from the skin should be reduced, the emulsified composition of the present invention preferably contains heparinoid or urea which are used as moisturizers in the treatment of xeroderma.

In particular, heparinoid is preferable since they are frequently used also in the treatment of atopic dermatitis.

From the viewpoint of preventing inflammation caused by dryness, it is preferable to add nonsteroidal anti-inflammatory agents which are widely used.

Among them, in view of safety, it is particularly preferable to use menthol, camphor, dipotassium glycyrrhizinate and allantoin that are widely used.

In view of use as antipruritic drugs for itchy dry skin, it is preferable to use crotamiton, diphenhydramine and salts thereof that are used as antipruritic agents to ease itchiness.

In particular, it is preferable to add crotamiton that is widely used.

Where necessary, the emulsified compositions of the present invention may contain other components generally used in the field of drugs, quasi drugs or cosmetics, in amounts and qualities that do not deteriorate properties such as storage stability and viscosity and do not impair the effects of the present invention. Such components include bases, preservatives, pH adjusters, stabilizers, irritation-reducing agents, antiseptics, coloring agents, dispersing agents, perfumes, and low-irritation surfactants other than lecithin. These components may be used singly, or two or more kinds may be used in any combination. The amounts of these components are not particularly limited as long as the effects of the present invention are achieved, but are desirably determined as appropriate within the pharmaceutically acceptable upper content limits.

The base materials include:

hydrocarbons such as paraffin, ozokerite, ceresin, hard fat, microcrystalline wax, squalane (synthetic and plant-derived), α-olefin oligomers, liquid paraffin, light isoparaffin, liquid isoparaffin and polyethylene powder;

fatty acids such as lauric acid, myristic acid, palmitic acid, stearic acid, behenic acid, isostearic acid, oleic acid and linoleic acid;

tri-fatty acid glycerides such as glyceryl tri-2-ethylhexanoate (trioctanoin);

polymerized silicones such as highly polymerized methylpolysiloxane, dimethylpolysiloxane, dimethylsiloxane/methyl(polyoxyethylene)siloxane/methyl(polyoxypropylene)siloxane copolymer, dimethylsiloxane/methyl(polyoxyethylene)siloxane copolymer, dimethylsiloxane/methyl(polyoxypropylene)siloxane copolymer, polyoxyethylene/methylpolysiloxane copolymer, poly(oxyethylene/oxypropylene)/methylpolysiloxane copolymer, dimethylsiloxane/methylcetyloxysiloxane copolymer, dimethylsiloxane/methylstearoxysiloxane copolymer, alkyl acrylate copolymer methylpolysiloxane esters, crosslinked methylpolysiloxane, crosslinked methylphenylpolysiloxane, crosslinked polyether-modified silicones, crosslinked alkyl polyether-modified silicones and crosslinked alkyl-modified silicones;

glycol acetates such as ethylene glycol monoacetate, ethylene glycol diacetate, triethylene glycol diacetate, hexylene glycol diacetate and 2-methyl-2-propene-1,1-diol diacetate;

glycol esters such as triethylene glycol divalerate, 2,2,4-trimethyl-1,3-pentanediol monoisobutyrate and 2,2,4-trimethyl-1,3-pentanediol diisobutyrate;

glycol acrylates such as ethylene glycol diacrylate, diethylene glycol diacrylate, propylene glycol monoacrylate, 2,2-dimethyl-trimethylene glycol diacrylate and 1,3-butylene glycol diacrylate;

glycol dinitrates such as ethylene glycol dinitrate, diethylene glycol dinitrate, triethylene glycol dinitrate and propylene glycol dinitrate;

ether compounds such as 2,2′-[1,4-phenylenedioxy]diethanol, dioxane and poly(butylene glycol adipate);

lower alcohols such as ethanol and isopropanol;

higher alcohols such as cetanol, stearyl alcohol, behenyl alcohol, cetostearyl alcohol, hexyldecanol, isostearyl alcohol, octyl dodecanol, oleyl alcohol, decyl tetradecanol and myristyl alcohol;

polyhydric alcohols (excluding glycerin) such as ethylene glycol, propylene glycol, 1,3-butylene glycol, 1,2-pentanediol and 1,2-hexanediol;

diethylene glycol alkyl ethers such as diethylene glycol monoethyl ether;

macrogol;

esters such as isopropyl myristate, octyldodecyl myristate, isopropyl palmitate, cetyl palmitate, glyceryl tri-2-ethylhexylate, glyceryl monostearate and medium chain triglyceride;

polyoxyethylene alkyl ethers such as polyoxyethylene behenyl ether; and

vegetable oils such as olive oil.

The preservatives include benzoic acid, sodium benzoate, dehydroacetic acid, sodium dehydroacetate, isobutyl paraoxybenzoate, isopropyl paraoxybenzoate, butyl paraoxybenzoate, ethyl paraoxybenzoate, propyl paraoxybenzoate, benzyl paraoxybenzoate, methyl paraoxybenzoate, phenoxyethanol and the like.

The pH adjusters include inorganic acids such as hydrochloric acid, sulfuric acid, phosphoric acid, polyphosphoric acid and boric acid; organic acids such as lactic acid, acetic acid, citric acid, tartaric acid, malic acid, succinic acid, sodium succinate, oxalic acid, gluconic acid, fumaric acid, propionic acid, acetic acid, aspartic acid, epsilon-aminocaproic acid, glutamic acid and aminoethylsulfonic acid; gluconolactone; ammonium acetate; inorganic bases such as sodium hydrogencarbonate, sodium carbonate, potassium hydroxide, sodium hydroxide, calcium hydroxide and magnesium hydroxide; organic bases such as monoethanolamine, triethanolamine, diisopropanolamine, triisopropanolamine and lysine and the like.

The low-irritation surfactants other than lecithin include polyoxyethylene alkyl ethers such as polyoxyethylene behenyl ether, polyoxyethylene stearyl ether and polyoxyethylene cetyl ether; polyoxyethylene polyoxypropylene alkyl ethers such as polyoxyethylene polyoxypropylene cetyl ether; polyoxyethylene hydrogenated castor oil; sorbitan fatty acid esters such as sorbitan sesquioleate and sorbitan monostearate; polyoxyethylene sorbitan fatty acid esters such as polyoxyethylene sorbitan stearate; polyoxyethylene fatty acid esters such as polyoxyl stearate; macrogol stearate; lanolin alcohol and the like.

The emulsified compositions of the present invention contain the components (A) to (E) in the specified amounts, and the balance thereof is excellent and the internal phase of the emulsified composition has a specific average particle diameter as will be described later. Consequently, the compositions have good sense of use while containing a sufficient amount of petrolatum enough to decrease the evaporation of water from the skin, can supply sufficient water to the skin, have sustainable and high moisture-retaining properties, and show low skin irritation and high storage stability. Further, the compositions repel ticks though they contain glycerin having a tick-attracting effect.

In the emulsified composition of the present invention, from the viewpoint of achieving the above effects, the preferred amounts of the components (A) to (E) are 10 to 30 wt % for the petrolatum (A), 0.1 to 5 wt % for the lecithin (B), 1 to 30 wt % for the glycerin (C), 40 to 75 wt % for the water (D), and 0.0001 to 5 wt % for the water-soluble polymer (E) based on the whole emulsified composition (100 wt %). The more preferred amounts of the components (A) to (E) are 12 to 27 wt % for the petrolatum (A), 0.5 to 3 wt % for the lecithin (B), 5 to 25 wt % for the glycerin (C), 60 to 75 wt % for the water (D), and 0.01 to 3 wt % for the water-soluble polymer (E) based on the whole emulsified composition (100 wt %). The particularly preferred amounts of the components (A) to (E) are 15 to 25 wt % for the petrolatum (A), 1.2 to 2 wt % for the lecithin (B), 10 to 20 wt % for the glycerin (C), 60 to 70 wt % for the water (D), and 0.01 to 1 wt % for the water-soluble polymer (E) based on the whole emulsified composition (100 wt %).

### [Average Particle Diameter]

The internal phase of the emulsified composition of the present invention has an average particle diameter of not more than 5000 nm, preferably not more than 2000 nm, and particularly preferably not more than 1000 nm as measured based on Mie theory. The lower limit of the average particle diameter is not particularly limited, but is generally 50 nm, preferably 100 nm, and particularly preferably 200 nm.

Average particle diameters exceeding 5000 nm are not preferable from the viewpoints of stability, sense of use and moisture-retaining properties of the emulsified composition of the present invention as will be demonstrated in Test Examples later. Obtaining emulsified compositions that have an internal phase average particle diameter of less than 50 nm involves large amounts of energy and often requires that surfactants having high emulsifying power but being highly irritating should be used to emulsify the compositions, not being preferable in terms of cost and irritancy reduction.

In detail, the average particle diameter which can be measured based on the Mie theory is the average particle diameter of an internal phase of an emulsified composition that is comprised of the petrolatum (A), lecithin (B), glycerin (C) and water (D).

Emulsified composition is an emulsion. As used herein, the term emulsion refers to a system formed of two immiscible liquids in which one of the liquids forms small droplets and is dispersed in the other liquid. The internal phase of the emulsified composition refers to such small droplets.

The average particle diameter means a median diameter that is obtained by automatically calculating the particle size distribution based on the Mie theory with scattered light data measured by batch measurement method using a laser diffraction/scattering particle size distribution analyzer (for example, LA-920 manufactured by HORIBA Ltd.). The median diameter is a particle diameter corresponding to cumulative 50% in frequency distribution.

The Mie theory cannot determine the average particle diameter of the internal phase of the emulsified composition according to the present invention which is obtained by adding the water-soluble polymer (E) to the emulsified composition comprised of the components (A), (B), (C) and (D). But it can be measured by a dynamic light scattering method based on the dynamic light scattering theory. The Mie theory calculates the particle size distribution by observing and analyzing the intensity distribution of scattered light, while the dynamic light scattering theory calculates the particle size distribution by observing and analyzing the fluctuation in intensity distribution by the interference of scattered light.

The average particle diameter of the internal phase of the emulsified composition of the present invention as measured by a dynamic light scattering method based on the dynamic light scattering theory is not more than 800 nm, preferably not more than 700 nm, and particularly preferably not more than 500 nm. If the average particle diameter exceeds 800 nm, the emulsified compositions deteriorate sense of use and stability. The lower limit of the average particle diameter is not particularly limited, but is generally 10 nm, preferably 50 nm, more preferably 100 nm, and particularly preferably 150 nm. Similar to the compositions comprised of the components (A), (B), (C) and (D), the emulsified composition according to the present invention is an emulsion. The words “the internal phase of the emulsified composition” means small droplets where a system is formed of two immiscible liquids in which one of the liquids forms small droplets and is dispersed in the other liquid.

This average particle diameter of the internal phase of the emulsified composition means a median diameter that is obtained by automatically calculating the particle size distribution based on the dynamic light scattering theory with scattered light data measured by batch measurement method using a laser diffraction/scattering particle size distribution analyzer (for example, FPAR-1000 manufactured by OTSUKA ELECTRONICS CO., LTD.). The median diameter is a particle diameter corresponding to cumulative 50% in frequency distribution.

### [Methods for Preparing Emulsified Composition]

The emulsified composition of the present invention may be prepared by any methods without limitation. For example, a mixture that contains the components (A) to (E) which constitute the emulsified composition of the present invention and other components such as active ingredients and base materials as required may be emulsified to afford an emulsified composition according to the present invention.

In an exemplary emulsifying method, a mixture that contains the components (A) to (E) which constitute the emulsified composition of the present invention and other components such as active ingredients and base materials as required is emulsified by great energy with an emulsifying machine capable of applying higher energy than a homomixer (for example, a microfluidizer, an ultrasonic emulsifier, a high pressure emulsifier (a high pressure homogenizer)).

This emulsifying treatment provides an emulsified composition of the present invention which has an average particle diameter satisfying the foregoing range as measured by a dynamic light scattering method based on the dynamic light scattering theory.

The whole amounts of the components (A) to (E) forming the emulsified composition of the invention and other components such as active ingredients and base materials may be emulsified together. Alternatively, part of the components for the emulsified composition may be emulsified and the remaining part may be thereafter added. Whether the emulsifying treatment involves the whole amounts together or part of the components followed by addition of the remaining part may be determined appropriately depending on the raw materials used.

The emulsified composition obtained as described above may be any of oil-in-water emulsion or water-in-oil emulsion, and is preferably oil-in-water emulsion. Generally, the compositions tend to be oil-in-water emulsion when the amount of the water (D) is 40 wt % or more based on 100 wt % of the emulsified composition.

### [Properties, Use, Etc. of Emulsified Composition]

The emulsified composition of the present invention may be prepared in various forms including creams, emulsion liquids and gel emulsions. Among them, as an embodiment of the present invention, emulsion liquids are preferable because of easy application to large area.

The viscosity of the emulsified compositions of the present invention at 25° C. is not particularly limited, but is generally in the range of 500 to 30000 mPa·s. The viscosity of the emulsified compositions of the present invention at 25° C. is preferably in the range of 800 to 10000 mPa·s, more preferably 900 to 7000 mPa·s, and particularly preferably 1000 to 5000 mPa·s.

If the viscosity is higher than 30000 mPa·s, the sense of use such as spreadability on skin (applicability) or stickiness may be poor. Viscosities less than 500 mPa·s may cause poor storage stability.

Herein, the viscosity is measured with respect to an emulsified composition placed in a 50 ml glass screw cap bottle with use of a BL viscometer (manufactured by TOKI SANGYO CO., LTD.) while selecting a rotor and a rotation speed that are most appropriate for the viscosity.

In detail, the viscosity is a value determined as follows. When the viscosity of the emulsified composition is in the range of 500 mPa·s to less than 4500 mPa·s, it is a value measured after an M2 rotor is rotated at 6 rpm and 25° C. for one minute. When the viscosity of the emulsified composition is in the range of 4500 mPa·s to less than 18000 mPa, it is a value measured after an M3 rotor is rotated at 6 rpm and 25° C. for one minute. When the viscosity of the emulsified composition is in the range of 18000 mPa·s to 30000 mPa·s, it is a value measured after an M4 rotor is rotated at 12 rpm and 25° C. for one minute.

In the event that the measurement of viscosity is infeasible because of slippage of the emulsified composition or the viscosity exceeding the upper measurement limit, the value measured as described below is adopted as viscosity as long as the hardness is not more than 1000 g. The hardness of the emulsified composition herein is a maximum value that is measured, while the emulsified composition is placed in a 50 g plastic jar, with a rheometer on the condition of T. speed (UP) of 2 cm/min and φ20 (compressive elasticity) adopter until the bottom of the adopter advances 1 cm.

The dosage and usage of the emulsified composition of the present invention to integuments are not particularly limited. The compositions may be usually applied to integuments such as skin several times a day each in an appropriate amount.

The emulsified composition of the present invention can be used for the treatment, prevention or improvement of various symptoms.

Examples of the use of the emulsified composition of the present invention include use for the treatment, prevention or improvement of a symptom caused by skin dryness and/or a disease that shows a symptom caused by skin dryness.

Exemplary symptoms caused by skin dryness include desquamation of skin surface, powdered skin, scuffing, dryness, cracks, chapped skin, keratinization on elbows, knees, heels, ankles or the like, fine facial wrinkles, hardened skin, rough hands and fingers, itching, dry skin, sensitive skin, skin rashes, erythema, atopic skin and rough skin.

Exemplary diseases showing the above symptoms include xeroderma, senile xerosis, ichthyosis vulgaris (scaly skin), infantile dry skin, atopic dermatitis, allergic dermatitis, asteatotic eczema, sensitive skin, seasonal xeroderma, aquagenic pruritus and housewives' eczema.

In general, dry skin has lowered its barrier function. The emulsified composition of the present invention has excellent moisture-retaining properties and can hydrate the skin, being useful for the treatment, prevention or improvement of dry skin. Furthermore, the emulsified composition of the present invention may increase the barrier function of the skin. Accordingly, the emulsified composition of the present invention has various effects, for example, to condition the skin, to improve the skin texture, to keep the skin healthy, and to protect the skin.

The emulsified composition of the present invention has a tick-repelling effect and is therefore useful for the treatment, prevention or improvement of scabies that is a skin infection evoked by ticks as well as itching, redness and eczema on the skin directly caused by bites or stings of tick.

It is known that atopic dermatitis symptoms repeatedly remit and exacerbate even when the patients are treated with steroids or the like. One reason for this is that many of the atopic dermatitis patients have allergy to ticks. Ticks are also an allergen for many skin diseases other than atopic dermatitis. It is very important to break off contact with tick allergens for the treatment of the diseases or symptoms. Since the emulsified composition of the present invention possesses a tick-repelling effect, it is useful for the treatment, prevention or improvement of symptoms evoked by skin dryness that are worsened by contact with ticks.

The emulsified composition according to the present invention as described above may be used in the manufacture of medicaments or cosmetics for the treatment, prevention or improvement of a symptom caused by skin dryness and/or a disease that shows a symptom caused by dry skin.

### EXAMPLES

The present invention will be described in detail hereinbelow without limiting the scope of the invention. Blending amounts described hereinbelow indicate wt % unless otherwise specified.

Two kinds of average particle diameters, and viscosity of emulsified compositions of Examples and Comparative Examples below were measured by the following methods.

### <Average Particle Diameter of Internal Phase of Emulsified Composition Formed of Petrolatum, Lecithin, Glycerin and Water>

Analyzer LA-920 manufactured by HORIBA Ltd. was used as a measuring device, and a batchwise measurement method was adopted.

A sample that had been emulsified by high pressure treatment was diluted with purified water ten times (weight ratio). Several droplets of the diluted sample were dropped to a glass cell (approximately 10 ml) filled with purified water, and the mixture was stirred. A 632.8 nm He—Ne laser beam was applied to the sample, and the scattered light was analyzed. From the scattered light data obtained, the particle size distribution was automatically calculated based on Mie theory to give an average particle diameter (a median diameter). The temperatures of the purified water and the surrounding environment were both approximately 25° C.

### <Average Particle Diameter of Internal Phase of Emulsified Composition Formed of Petrolatum, Lecithin, Glycerin, Water and Water-Soluble Polymer>

Analyzer FPAR-1000 was used as a measuring device, and a batchwise measurement method was adopted.

A sample that had been emulsified by high pressure treatment was diluted with purified water ten times (weight ratio). Approximately 5 ml of the diluted sample was added to a glass vial and was stirred. The vial was set in a cell. A 650.0 nm semiconductor laser beam was applied to the sample, and the scattered light was analyzed. From the scattered light data obtained, the particle size distribution was automatically calculated based on the dynamic light scattering theory to give an average particle diameter (a median diameter). The temperatures of the purified water and the surrounding environment were both approximately 25° C.

### <Measurement of Viscosity>

A BL viscometer (manufactured by TOKI SANGYO CO., LTD.) was used as a measuring device.

The emulsified composition was placed in a 50 ml glass screw cap bottle, a rotor and a rotation that were most appropriate for the viscosity were selected, and the viscosity was measured at 25° C. after one minute had passed.

The rotor and rotation were M2 rotor and 6 rpm when the viscosity of the emulsified composition was in the range of 500 mPa·s to less than 4500 mPa·s, and M3 rotor and 6 rpm when the viscosity of the emulsified composition was in the range of 4500 mPa·s to less than 18000 mPa·s.

### Examples 1 to 3

Petrolatum and lecithin were mixed together and molten by heating. The temperature of the obtained mixture was brought to 75 to 80° C. Separately, part of water was mixed with glycerin, and the mixture was heated to 75 to 80° C. The mixture was then added to the petrolatum/lecithin mixture, and these were preliminarily mixed together with a homomixer. The resultant mixture was held within a temperature of 75 to 80° C. and emulsified by high pressure homogenizer treatment at 500 bar. The emulsion was cooled. The emulsion was then mixed with xanthan gum dissolved in the remaining part of water. In this manner, emulsified compositions of Examples 1 to 3 with makeups as shown in Table 1 were prepared.

The average particle diameter of the internal phase of the emulsified composition before adding the xanthan gum dissolved in the remaining part of water was measured (based on Mie theory), and the average particle diameter of the internal phase of the emulsified composition after adding the xanthan gum dissolved in the remaining part of water was measured (by a dynamic light scattering method based on the dynamic light scattering theory). The results are also shown in Table 1.

TABLE 1

| | Ex. 1 | Ex. 2 | Ex. 3 |
|---|---|---|---|
| Petrolatum | 10 | 15 | 30 |
| Glycerin | 15 | 20 | 15 |
| Lecithin*¹ | 1.25 | 1.5 | 3 |
| Xanthan gum | 0.2 | 0.2 | 0.2 |
| Water | 73.55 | 63.3 | 51.8 |
| Average particle diameter (nm, measured | 764 | 689 | 685 |
| based on Mie theory) | | | |
| Average particle diameter (nm, measured | 434 | 277 | 372 |
| based on dynamic light scattering theory) | | | |

*¹NIKKOL Lecinol S-10 (manufactured by Nikko Chemicals Co., Ltd.)

It was assumed that there was no difference in average particle diameter of the internal phase of the emulsified composition between before and after the xanthan gum dissolved in the remaining part of water was admixed. To confirm this assumption, the average particle diameters were measured based on electron micrographs.

The measurement of the average particle diameters based on electron micrographs was conducted as follows. With respect to the emulsified composition of Example 2, electron micrographs of the emulsified composition were taken before and after the xanthan gum dissolved in the remaining part of water was admixed therewith. Arbitrary 50 particles (that did not overlap with any another particles) were selected and the diameters thereof were measured. The median diameter was obtained as average particle diameter. The results are shown in Table 2.

TABLE 2
Average particle diameter (nm)

| Before mixing xanthan gum | After mixing xanthan gum |
|---|---|
| 189 | 190 |

The measurement based on Mie theory provided the average particle diameter of the emulsified composition before admixing the xanthan gum dissolved in the remaining part of water. The measurement by a dynamic light scattering method based on the dynamic light scattering theory provided the average particle diameter of the emulsified composition after admixing the xanthan gum dissolved in the remaining part of water. The measurements of the average particle diameters based on the electron micrographs confirmed that the average particle diameter of the emulsified composition had not changed before and after xanthan gum dissolved in the remaining part of water was admixed. Although greatly differing values resulted from the two measurements (one based on Mie theory and the other by a dynamic light scattering method based on the dynamic light scattering theory), this great difference was ascribed to differing theories on which the measurement was based.

The viscosity of the emulsified composition of Example 1 was 920 mPa·s. The viscosities in Examples 2 and 3 were 1080 mPa·s and 1120 mPa·s, respectively.

### Comparative Examples 1 to 5

Emulsified compositions of Comparative Examples 1 and 2 were prepared in the same manner as in Examples 1 to 3, except that the components were used in blending amounts shown in below Table 3.

An emulsified composition of Comparative Example 3 was prepared in the same manner as in Examples 1 to 3, except that glycerin was not added and the components were added in blending amounts as shown in below Table 3. An emulsified composition of Comparative Example 4 was prepared in the same manner as in Examples 1 to 3, except that xanthan gum was not added and the components were added in blending amounts as shown in below Table 3.

An emulsified composition of Comparative Example 5 was prepared as follows. Petrolatum and lecithin were mixed together and molten by heating. The temperature of the obtained mixture was brought to 75 to 80° C. Separately, part of water was mixed with glycerin, and the obtained mixture was heated to 75 to 80° C. The mixture was added to the petrolatum/lecithin mixture and emulsified with a homomixer. The emulsion was cooled. The emulsion was then mixed with xanthan gum dissolved in the remaining part of water to give an emulsified composition.

The makeups and average particle diameters of the emulsified compositions of Comparative Examples 1 to 5 are shown in Table 3. In Comparative Examples 1 to 3 and 5, the average particle diameter of the internal phase of the emulsified composition was measured (based on Mie theory) before admixing the xanthan gum dissolved in the remaining part of water, and the average particle diameter of the emulsified composition was measured (by a dynamic light scattering method based on the dynamic light scattering theory) after admixing the xanthan gum dissolved in the remaining part of water. In Comparative Example 4, the average particle diameter of the internal phase of the emulsified composition was measured based on Mie theory and by a dynamic light scattering method based on the dynamic light scattering theory.

TABLE 3

| | Comp. | Comp. | Comp. | Comp. | Comp. |
|---|---|---|---|---|---|
| | Ex. 1 | Ex. 2 | Ex. 3 | Ex. 4 | Ex. 5 |
| Petrolatum | 5 | 45 | 15 | 15 | 15 |
| Glycerin | 20 | 15 | — | 20 | 20 |
| Lecithin*¹ | 0.5 | 4.5 | 1.5 | 1.5 | 1.5 |
| Xanthan gum | 0.2 | 0.2 | 0.2 | — | 0.2 |
| water | 74.3 | 35.3 | 83.3 | 63.5 | 63.3 |
| Average particle | 1075 | 737 | 1874 | 688 | 9716 |
| diameter (nm, measured | | | | | |
| based on Mie theory) | | | | | |
| Average particle | 312 | 377 | 618 | 977 | 890 |
| diameter (nm, measured | | | | | |
| based on dynamic light | | | | | |
| scattering theory) | | | | | |

*¹NIKKOL Lecinol S-10 (manufactured by Nikko Chemicals Co., Ltd.)

Similar to Example 2, it was assumed that there was no difference in average particle diameter of the internal phase of each of the emulsified compositions of Comparative Examples 1 to 3 and 5 between before and after the xanthan gum dissolved in the remaining part of water was admixed.

The viscosity of the emulsified composition of Comparative Example 1 was 785 mPa·s. The viscosities in Comparative Examples 2 and 3 were 12600 mPa·s and 1020 mPa·s, respectively.

### Test Example 1

### Evaluation of Moisture-Retaining Properties (Amount of Water Evaporation)

Water (10 ml) was placed in a 50 ml screw cap bottle, and the bottle was covered with synthetic leather (product name: SUPPLALE PBZ 13001, Idemitsu Technofine Co., Ltd.). The amount of water evaporation (the amount of evaporation per unit area and unit time before the application of the emulsified composition) was measured with a water evaporation monitor (AS-TW2 manufactured by ASAHI BIOMED). Thereafter, the emulsified composition (8 mg) of any one of Examples 1 to 3 and Comparative Examples 1, 2 and 5 was applied to the synthetic leather and was allowed to stand at room temperature for 20 hours. The amount of water evaporation (after 20 hours after the application) was measured with the water evaporation monitor (AS-TW2, manufactured by ASAHI BIOMED). This standing for 20 hours was to avoid measuring the evaporation of water from the emulsified composition itself when measured immediately after the application.

The water evaporation inhibition rates (%) of the emulsified compositions from Examples 1 to 3 and Comparative Examples 1, 2 and 5 are shown in Table 4. The water evaporation inhibition rate was calculated by the following equation:

Water evaporation inhibition rate (%)=(1−(water evaporation amount after 20 hours after application/water evaporation amount before application))*100

TABLE 4

| | Water evaporation inhibition rate (%) |
|---|---|
| Ex. 1 | 60.4 |
| Ex. 2 | 74.7 |
| Ex. 3 | 71.8 |
| Comp. Ex. 1 | 40.8 |
| Comp. Ex. 2 | 58.5 |
| Comp. Ex. 5 | 61.3 |

It is generally known that petrolatum increases skin occlusion and thereby shows an effect of decreasing the evaporation of water from the skin. This effect of petrolatum agrees with the above result in which the Examples 1 to 3 containing more petrolatum achieved higher water evaporation inhibition rates as compared with the Comparative Example 1 containing less petrolatum. However, Comparative Example 2 in which the petrolatum content was further increased resulted in a lower water evaporation inhibition rate compared to Examples 1 to 3. These results indicate that simply increasing the petrolatum content does not inhibit the water evaporation and that markedly high effects of water evaporation inhibition are achieved when the emulsified composition of the present invention contains petrolatum in the specific amount range.

Comparing the results of Example 2 and Comparative Example 5 in which the respective emulsified compositions had an identical makeup showed that the water evaporation inhibition effects were improved by decreasing the average particle diameter of the internal phase of the emulsified composition.

### Test Example 2

### Evaluation of Emulsion Stability

Approximately 15 g of each of the emulsified compositions from Examples 1 to 3 and Comparative Examples 1 to 5 was placed in a respective 20 ml transparent screw cap bottle, and was stored at 60° C. for 2 days. They were then visually observed for the occurrence of transparent phase separation. The results are shown in Table 5 in which A indicates no phase separation and B indicates transparent phase separation.

TABLE 5

| | 60° C., 2 days |
|---|---|
| Ex. 1 | A |
| Ex. 2 | A |
| Ex. 3 | A |
| Comp. Ex. 1 | B |
| Comp. Ex. 2 | A |
| Comp. Ex. 3 | A |
| Comp. Ex. 4 | A |
| Comp. Ex. 5 | B |

No phase separation was observed in the Examples 1 to 3 and Comparative Examples 2 to 4. However, the Comparative Example 1 in which the petrolatum content was lower as compared with those in Examples 1 to 3 had phase separation, showing poor emulsion stability. Comparing the results of Example 2 and Comparative Example 5 in which the respective emulsified compositions had an identical makeup showed that the emulsion stability was improved by decreasing the average particle diameter of the internal phase of the emulsified composition.

### Test Example 3

### Evaluation of Sense of Use

The emulsified compositions from Examples 1 to 3 and Comparative Examples 1 to 5 were applied to the arms of ten test subjects and were evaluated in terms of ‘greasiness’, ‘spreadability’, ‘gloss’ and ‘moistness’, on the three scales: ‘satisfied’, ‘normal’ and ‘unsatisfied’.

The evaluation results are shown in Table 6 in which A indicates that 8 or more test subjects answered satisfied or normal, B indicates that 6 or 7 test subjects answered satisfied or normal, and C indicates that 5 or more test subjects answered unsatisfied.

TABLE 6

| | Greasiness | Spreadability | Gloss | Moistness |
|---|---|---|---|---|
| Ex. 1 | A | A | A | A |
| Ex. 2 | A | A | A | A |
| Ex. 3 | A | A | A | A |
| Comp. | B | A | C | C |
| Ex. 1 | | | | |
| Comp. | C | C | B | A |
| Ex. 2 | | | | |
| Comp. | A | A | A | B |
| Ex. 3 | | | | |
| Comp. | A | A | A | C |
| Ex. 4 | | | | |
| Comp. | A | A | A | B |
| Ex. 5 | | | | |

The Examples 1 to 3 were evaluated A in all the items, in contrast to no Comparative Examples 1 to 5 that were evaluated A in all the items.

In particular, the Comparative Example 1 containing less petrolatum showed low scores in ‘gloss’ and ‘moistness’, and the Comparative Example 2 containing much petrolatum showed low scores in ‘greasiness’ and ‘spreadability’. The Comparative Example 4 containing no water-soluble polymer showed a low score in ‘moistness’, and thereby it was confirmed that addition of the water-soluble polymers gave moistness. Compared to Examples 1 to 3, ‘moistness’ was evaluated to be inferior in Comparative Example 3 involving no glycerin and Comparative Example 5 in which the internal phase of the emulsified composition had a large average particle diameter.

The emulsified compositions of Examples 1 to 3 were confirmed to be nonirritating.

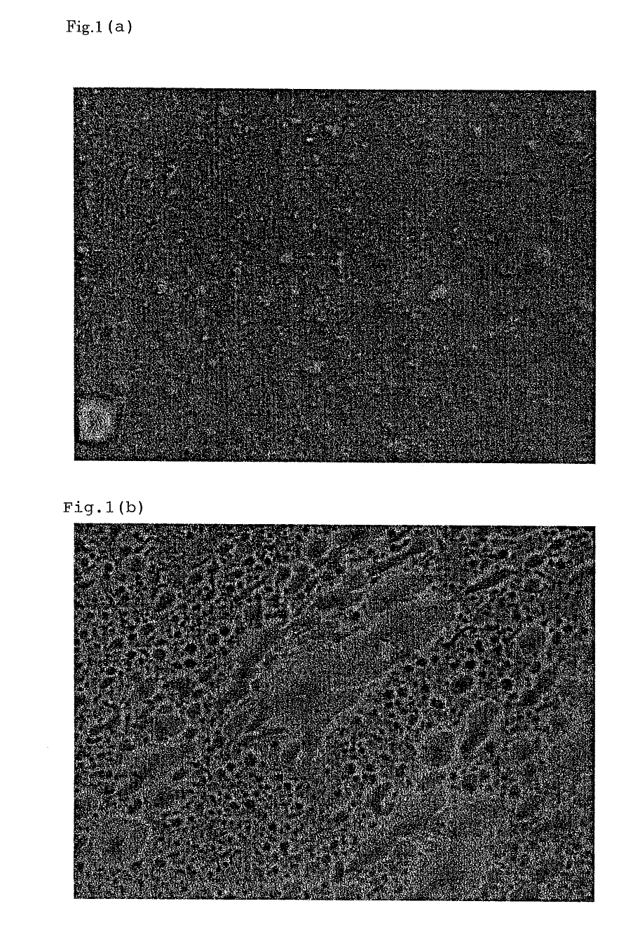

FIG. 1 shows micrographs of the emulsified compositions of Example 2 and Comparative Example 5 applied to a glass plate.

As seen in FIG. 1, the emulsified composition according to the present invention was confirmed to be applied more homogeneously as compared with the emulsified composition of Comparative Example 5 having a larger average particle diameter of the internal phase.

### Test Example 4

### Evaluation of Tick-Repelling Properties

In this test example, emulsified compositions given in Table 7 below were used.

TABLE 7

| | | Comp. | Comp. |
|---|---|---|---|
| | Ex. 4 | Ex. 6 | Ex. 7 |
| Petrolatum | 20 | 20 | 20 |
| Glycerin | 20 | 20 | 20 |
| Lecithin*¹ | 2 | 2 | 2 |
| Xanthan gum | 0.5 | — | 0.5 |
| Water | 57.5 | 58 | 57.5 |
| Average particle diameter (nm, | 868 | 868 | 11867 |
| measured based on Mie theory) | | | |
| Average particle diameter (nm, | 361 | 516 | 1186 |
| measured based on dynamic | | | |
| light scattering theory) | | | |

*¹NIKKOL Lecinol S-10 (manufactured by Nikko Chemicals Co., Ltd.)

The emulsified composition of Example 4 was prepared in the same manner as in Examples 1 to 3, except that the components were blended in amounts as shown in Table 7. The emulsified composition of Comparative Example 6 was prepared in the same manner as in Examples 1 to 3, except that xanthan gum was not added and the components were blended in amounts as shown in Table 7. The emulsified composition of Comparative Example 7 was prepared in the same manner as in Comparative Example 5, except that the components were blended in amounts as shown in Table 7.

In Example 4 and Comparative Example 7, the average particle diameter of the internal phase of the emulsified composition was measured (based on Mie theory) before admixing the xanthan gum dissolved in the remaining part, and the average particle diameter of the emulsified composition was measured (by a dynamic light scattering method based on the dynamic light scattering theory) after admixing the xanthan gum dissolved in the remaining part of water. In Comparative Example 6, the average particle diameter of the internal phase of the emulsified composition was measured by a method based on Mie theory and by a dynamic light scattering method based on the dynamic light scattering theory. The results are shown in Table 7 above.

Similar to Example 2, it was assumed in Example 4 and Comparative Example 7 that there was no difference in average particle diameter of the internal phase of the emulsified composition between before and after the xanthan gum dissolved in the remaining part of water was admixed.

The viscosity of the emulsified composition of Example 4 was 6120 mPa·s.

Each (20 mg) of the emulsified compositions shown in Table 7 was uniformly applied to circular filter paper having a diameter of 4 cm. The filter paper was laid on the inner bottom surface of a glass Petri dish having an inner diameter of 4 cm. A tick feeding medium (50 mg; a 1:1 mixture of a powdery feed for small animals (MF, manufactured by Oriental Yeast Co., Ltd.) and an officinal dry yeast (ASAHI BREWERIES, LTD.)) was placed in the center of the filter paper. A tick medium containing Dermatophagoides pteronyssinus (population: approximately 10000) was uniformly laid on the inner bottom surface of a glass Petri dish having a diameter of 9 cm, and the 4 cm diameter Petri dish having the emulsified composition described above was disposed in the center of the bottom surface. The Petri dish was placed in a sealable food storage container (capacity: 8.5 L), and a saturated saline solution was added to the bottom surface in the sealable container. The humidity was adjusted to approximately 75% RH.

The test device manufactured as described above was stored in a light-shielded room that was temperature-controlled at 25° C. After 24 hours, the number of ticks that had moved to the tick feeding medium and filter paper was counted. In all the tests, n=3. The control was the result obtained with 4 cm diameter circular filter paper without the application of the emulsified composition. The ticks on the tick feeding medium were collected by a saturated saline solution flotation method, and the ticks on the filter paper were collected by washing method. The number of them was counted with a stereomicroscope. The numbers of ticks that had moved to the filter paper and the tick-repelling rates are set forth in Table 8. The tick-repelling rate (%) was calculated based on the following equation:

Tick-repelling rate (%)=(control number of ticks that had moved−number of ticks that had moved)/control number of ticks that had moved*100

TABLE 8

| | | | | Total | |
|---|---|---|---|---|---|
| | Moved number | | | number of ticks | Tick-repelling |
| | 1 | 2 | 3 | that had moved | rate (%) |
| Ex. 4 | 1210 | 839 | 1171 | 3220 | 28.5 |
| Comp. Ex. 6 | 1050 | 1243 | 1383 | 3676 | 18.4 |
| Comp. Ex. 7 | 1264 | 1190 | 1511 | 3965 | 12.0 |
| Control | 1298 | 1431 | 1775 | 4505 | — |

The above results provide that the emulsified composition of Example 4 according to the present invention achieved higher tick-repelling effects than that of Comparative Example 7 having an identical makeup and a larger average particle diameter of the internal phase and that of Comparative Example 6 containing no water-soluble polymer.

### Test Example 5

### Evaluation of Moisture-Retaining Properties (Water Content in Horny Layer)

Four points on the inner side of forearms of three test subjects were marked with 2 cm×2 cm marks as testing sites. Approximately 8 mg of each of the emulsified compositions from Examples 1 to 3 and Comparative Examples 1 and 3 was applied to the testing sites. The water content in horny layer was measured before the application, and after 5 minutes, 30 minutes and 60 minutes from the application with SKICON-200 (manufactured by IBS, central electrode diameter: 2 mm, load: 10 g). Herein, the conductance of the horny layer was measured. Because water is conductive, the conductance is positively correlated with the water content.

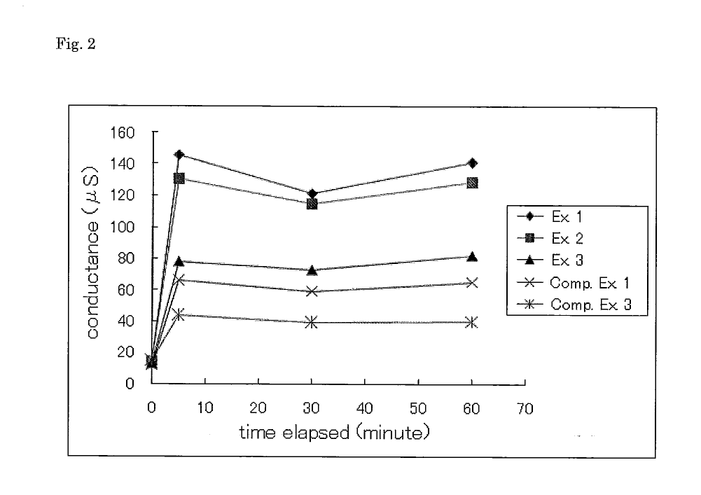

The ratio of the post-application conductance to the pre-application conductance (post-application conductance/pre-application conductance) was evaluated as B when it is less than 6 and was evaluated as A when it was 6 or more. The results are shown in Table 9 and FIG. 2.

TABLE 9

| | | | | | Ratio | | | | | |
|---|---|---|---|---|---|---|---|---|---|---|
| | Conductance (μS) | | | | to pre-application | | | Evaluation | | |
| | Before | After 5 | After 30 | After 60 | After 5 | After 30 | After 60 | After 5 | After 30 | After 60 |
| | application | minutes | minutes | minutes | minutes | minutes | minutes | minutes | minutes | minutes |
| Ex. 1 | 14 | 146 | 122 | 141 | 10.3 | 8.5 | 9.9 | A | A | A |
| Ex. 2 | 14 | 131 | 115 | 128 | 9.3 | 8.1 | 9.1 | A | A | A |
| Ex. 3 | 13 | 78 | 73 | 82 | 6.0 | 5.6 | 6.3 | A | B | A |
| Comp. | 11 | 66 | 59 | 65 | 5.8 | 5.2 | 5.7 | B | B | B |
| Ex. 1 | | | | | | | | | | |
| Comp. | 15 | 44 | 39 | 40 | 3.0 | 2.7 | 2.7 | B | B | B |
| Ex. 3 | | | | | | | | | | |

As evident from FIG. 2, the Examples 1 and 2 containing much water provided a high water content in horny layer and maintained the water content for 60 minutes. The emulsified composition of Example 3 contained less water and therefore provided a lower water content in horny layer than by those of Examples 1 and 2. In contrast, the emulsified composition of Comparative Example 1 containing more water than the emulsified composition of Example 1 provided a lower water content in horny layer than by that of Example 3, in spite of the larger amount of water added. This would be partly because the emulsified composition of Comparative Example 1 contained a small amount of petrolatum.

The emulsified composition of Comparative Example 3 did not contain glycerol and thus provided a very small water content in horny layer.

Formulation Examples are illustrated in Tables 10 to 13 below, but the scope of the present invention is not limited to them.

TABLE 10

| | Form. | Form. | Form. | Form. | Form. | Form. | Form. | Form. |
|---|---|---|---|---|---|---|---|---|
| | Ex. 1 | Ex. 2 | Ex. 3 | Ex. 4 | Ex. 5 | Ex. 6 | Ex. 7 | Ex. 8 |
| Petrolatum | 15 | 20 | 15 | 25 | 15 | 10 | 10 | 25 |
| Glycerin | 10 | 20 | 20 | 10 | 20 | 20 | 15 | 20 |
| Lecithin | 2 | 2 | 1.5 | 3 | 1.5 | 1.5 | 1.5 | 3 |
| Xanthan gum | 0.2 | 0.5 | — | 0.2 | — | — | — | — |
| Hydroxypropyl | — | — | 0.2 | — | — | — | 0.2 | — |
| methyl | | | | | | | | |
| cellulose | | | | | | | | |
| Hydroxyethyl | — | — | — | — | — | 0.3 | — | — |
| cellulose | | | | | | | | |
| Carboxyvinyl | — | — | — | — | 0.25 | — | — | 0.1 |
| polymer | | | | | | | | |
| Triethanolamine | — | — | — | — | 0.025 | — | — | 0.01 |
| Water | 72.8 | 57.5 | 63.3 | 61.8 | 63.225 | 68.2 | 73.3 | 51.89 |

TABLE 11

| | Form. Ex. 9 | Form. Ex. 10 | Form. Ex. 11 | Form. Ex. 12 | Form. Ex. 13 | Form. Ex. 14 | Form. Ex. 15 |
|---|---|---|---|---|---|---|---|
| Petrolatum | 15 | 15 | 20 | 20 | 20 | 15 | 15 |
| Glycerin | 20 | 20 | 15 | 15 | 15 | 20 | 15 |
| Lecithin | 1.5 | 1.5 | 2.5 | 2 | 2 | 1.5 | 1.5 |
| Xanthan gum | 0.2 | — | — | 0.15 | — | 0.2 | — |
| Carmellose sodium | — | 0.1 | — | — | 0.1 | — | 0.2 |
| Carboxyvinyl polymer | — | — | 0.5 | — | 0.2 | — | 0.5 |
| Prednisolone valerate | 0.15 | — | — | — | — | — | — |
| acetate | | | | | | | |
| Dexamethasone acetate | — | 0.025 | — | — | — | — | — |
| Crotamiton | — | — | 5 | — | — | — | 5 |
| Dipotassium | — | — | — | 0.1 | — | 0.05 | — |
| glycyrrhizinate | | | | | | | |
| Allantoin | — | — | — | — | 0.2 | 0.1 | — |
| Lidocaine | — | — | — | — | — | — | 2 |
| Diphenhydramine | — | — | — | — | — | — | 1 |
| Glycyrrhetinic acid | — | — | — | — | — | — | 0.2 |
| Urea | — | — | — | — | — | — | — |
| Monoammonium | — | — | — | — | — | — | — |
| glycyrrhizinate | | | | | | | |
| Tocopherol acetate | — | — | — | — | — | — | — |
| Heparinoid | — | — | — | — | — | — | — |
| Diphenhydramine | — | — | — | — | — | — | — |
| hydrochloride | | | | | | | |
| Sodium hyaluronate | — | — | — | — | — | — | — |
| pH adjuster | Appropriate | Appropriate | Appropriate | Appropriate | Appropriate | Appropriate | Appropriate |
| | amount | amount | amount | amount | amount | amount | amount |
| Water | Balance | Balance | Balance | Balance | Balance | Balance | Balance |
| Total | 100 | 100 | 100 | 100 | 100 | 100 | 100 |

TABLE 12

| | Form. Ex. 16 | Form. Ex. 17 | Form. Ex. 18 | Form. Ex. 19 | Form. Ex. 20 | Form. Ex. 21 | Form. Ex. 22 |
|---|---|---|---|---|---|---|---|
| Petrolatum | 15 | 15 | 15 | 20 | 20 | 15 | 15 |
| Glycerin | 15 | 15 | 15 | 15 | 15 | 20 | 20 |
| Lecithin | 1.5 | 1.5 | 1.5 | 2.5 | 3 | 3 | 2.5 |
| Xanthan gum | — | — | — | 0.2 | 0.2 | 0.2 | — |
| Carmellose sodium | 0.2 | 0.2 | 0.2 | — | 0.05 | 0.05 | 0.1 |
| Carboxyvinyl polymer | 0.5 | 0.5 | 0.5 | — | 0.3 | 0.3 | 0.3 |
| Prednisolone valerate | — | — | — | 0.15 | 0.15 | 0.15 | — |
| acetate | | | | | | | |
| Dexamethasone acetate | — | — | — | — | — | — | 0.025 |
| Crotamiton | 5 | — | 5 | 5 | 5 | 5 | 5 |
| Dipotassium | — | — | — | — | — | — | — |
| glycyrrhizinate | | | | | | | |
| Allantoin | — | — | — | — | — | 0.2 | 0.2 |
| Lidocaine | — | — | — | — | — | — | 1 |
| Diphenhydramine | 1 | — | 1 | — | — | — | — |
| Glycyrrhetinic acid | — | — | — | — | — | — | — |
| Urea | 10 | 20 | 20 | — | — | — | — |
| Monoammonium | 0.5 | 0.5 | 0.5 | — | — | — | — |
| glycyrrhizinate | | | | | | | |
| Tocopherol acetate | 0.5 | 0.5 | 0.5 | — | — | — | — |
| Heparinoid | — | — | — | — | — | — | — |
| Diphenhydramine | — | — | — | — | 1 | — | 1 |
| hydrochloride | | | | | | | |
| Sodium hyaluronate | — | — | — | — | — | — | — |
| pH adjuster | Appropriate | Appropriate | Appropriate | Appropriate | Appropriate | Appropriate | Appropriate |
| | amount | amount | amount | amount | amount | amount | amount |
| Water | Balance | Balance | Balance | Balance | Balance | Balance | Balance |
| Total | 100 | 100 | 100 | 100 | 100 | 100 | 100 |

TABLE 13

| | Form. Ex. 23 | Form. Ex. 24 | Form. Ex. 25 | Form. Ex. 26 | Form. Ex. 27 | Form. Ex. 28 |
|---|---|---|---|---|---|---|
| Petrolatum | 15 | 15 | 15 | 15 | 15 | 15 |
| Glycerin | 20 | 20 | 20 | 15 | 15 | 20 |
| Lecithin | 1.5 | 1.5 | 1.5 | 2 | 2 | 1.75 |
| Xanthan gum | 0.2 | 0.2 | 0.2 | 0.2 | — | 0.3 |
| Carmellose sodium | — | — | — | — | — | — |
| Carboxyvinyl polymer | — | — | — | — | 0.3 | — |
| Prednisolone valerate acetate | — | 0.15 | 0.15 | — | — | — |
| Dexamethasone acetate | — | — | — | — | — | — |
| Crotamiton | — | — | — | — | — | — |
| Dipotassium glycyrrhizinate | — | — | — | 0.05 | 0.05 | — |
| Allantoin | — | — | 0.2 | 0.1 | 0.1 | — |
| Lidocaine | — | — | — | — | — | — |
| Diphenhydramine | — | — | — | — | — | — |
| Glycyrrhetinic acid | — | — | — | — | — | — |
| Urea | — | — | — | — | — | — |
| Monoammonium glycyrrhizinate | — | — | — | — | — | — |
| Tocopherol acetate | — | — | — | — | — | — |
| Heparinoid | 0.3 | 0.3 | 0.3 | 0.1 | — | — |
| Diphenhydramine hydrochloride | — | — | — | — | — | — |
| Camphor | — | — | — | — | — | 10 |
| Menthol | — | — | — | — | — | 1.5 |
| Eucalyptus oil | — | — | — | — | — | 1.5 |
| Sodium hyaluronate | — | — | — | — | 0.05 | — |
| pH adjuster | Appropriate | Appropriate | Appropriate | Appropriate | Appropriate | Appropriate |
| | amount | amount | amount | amount | amount | amount |
| Water | Balance | Balance | Balance | Balance | Balance | Balance |
| Total | 100 | 100 | 100 | 100 | 100 | 100 |

1. An emulsified composition which comprises:
(A) 10 to 30 wt % of petrolatum,
(B) lecithin,
(C) glycerin,
(D) water and
(E) a water-soluble polymer; and
an internal phase of which has an average particle diameter of not more than 5000 nm.

2. The emulsified composition according to claim 1, which comprises (A) petrolatum, (B) lecithin, (C) glycerin, (D) water and (E) a water-soluble polymer,
wherein the content of the petrolatum (A) is 10 to 30 wt % relative to 100 wt % of the emulsified composition,
and the internal phase of the emulsified composition has an average particle diameter of not more than 800 nm as measured by a dynamic light scattering method based on the dynamic light scattering theory.

3. The emulsified composition according to claim 1, wherein the water-soluble polymer (E) is at least one polymer selected from the group consisting of cellulose polymers, vinyl polymers, acrylic acid polymers, plant polymers, microbial polymers, phospholipid polar group-containing polymers and mucopolysaccharides.

4. The emulsified composition according to claim 1, wherein the content of the glycerin (C) is 10 to 20 wt % relative to 100 wt % of the emulsified composition.

5. The emulsified composition according to claim 1, further comprising at least one active ingredient selected from the group consisting of nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents.

6. The emulsified composition according to claim 1, further comprising at least one active ingredient selected from the group consisting of nonsteroidal anti-inflammatory agents, steroids, local anesthetics, antipruritics and moisturizers.

7. The emulsified composition according to claim 1, which is used to treat, prevent or improve a symptom caused by skin dryness and/or a disease that shows a symptom caused by skin dryness.

8. A method for treating, preventing or improving a symptom caused by skin dryness and/or a disease that shows a symptom caused by skin dryness, comprising applying to skin an effective amount of the emulsified composition described in claim 1.

9. Use of the emulsified composition described in claim 1 in the manufacture of medicaments or cosmetics for treating, preventing or improving a symptom caused by skin dryness and/or a disease that shows a symptom caused by skin dryness.

10. The emulsified composition according to claim 2, wherein the water-soluble polymer (E) is at least one polymer selected from the group consisting of cellulose polymers, vinyl polymers, acrylic acid polymers, plant polymers, microbial polymers, phospholipid polar group-containing polymers and mucopolysaccharides.

11. The emulsified composition according to claim 2, wherein the content of the glycerin (C) is 10 to 20 wt % relative to 100 wt % of the emulsified composition.

12. The emulsified composition according to claim 3, wherein the content of the glycerin (C) is 10 to 20 wt % relative to 100 wt % of the emulsified composition.

13. The emulsified composition according to claim 2, further comprising at least one active ingredient selected from the group consisting of nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents.

14. The emulsified composition according to claim 3, further comprising at least one active ingredient selected from the group consisting of nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents.

15. The emulsified composition according to claim 4, further comprising at least one active ingredient selected from the group consisting of nonsteroidal anti-inflammatory agents, vitamins, whitening agents, anti-wrinkle agents, anti-inflammatory analgesics, antifungal agents, steroids, hair restorers, slimming agents, local anesthetics, antipruritics, antimicrobials, antivirals, keratin softeners, moisturizers, astringents, antioxidants, hair growth inhibitors, UV absorbents and UV scattering agents.

16. The emulsified composition according to claim 2, further comprising at least one active ingredient selected from the group consisting of nonsteroidal anti-inflammatory agents, steroids, local anesthetics, antipruritics and moisturizers.

17. The emulsified composition according to claim 3, further comprising at least one active ingredient selected from the group consisting of nonsteroidal anti-inflammatory agents, steroids, local anesthetics, antipruritics and moisturizers.

18. The emulsified composition according to claim 4, further comprising at least one active ingredient selected from the group consisting of nonsteroidal anti-inflammatory agents, steroids, local anesthetics, antipruritics and moisturizers.

19. The emulsified composition according to claim 2, which is used to treat, prevent or improve a symptom caused by skin dryness and/or a disease that shows a symptom caused by skin dryness.

20. The emulsified composition according to claim 3, which is used to treat, prevent or improve a symptom caused by skin dryness and/or a disease that shows a symptom caused by skin dryness.
